(19) United States
(12) Patent Application Publication
(10) Pub. No.: US 2026/0365605 A1
(43) Pub. Date: Nov. 19, 2026

(54) PROCESS OF CONTROLLING HEAVIES IN A RECYCLE CATALYST STREAM

(71) Applicant: Dow Technology Investments LLC, Midland, MI (US)

(72) Inventors: Deven Swapneshu Baser, Missouri City, TX (US); Yujun Liu, Pearland, TX (US); Glenn A. Miller, South Charleston, WV (US)

(21) Appl. No.: 19/119,825

(22) PCT Filed: Nov. 15, 2023
(86) PCT No.: PCT/US23/79790

§ 371 Date: Apr. 10, 2025

Related U.S. Application Data
(60) Provisional application No. 63/386,276, filed on Dec. 6, 2022.

(51) Int. Cl. C07C 45/82; C07C 45/50

(52) U.S. Cl. CPC C07C 45/82; C07C 45/50; C07C 2531/18

(57) ABSTRACT

A process of controlling heavies in a recycle catalyst stream, particularly, for use in a continuous hydroformylation process of converting an olefin with synthesis gas in the presence of a hydroformylation catalyst to form an aldehyde product stream with subsequent separation of the catalyst for recycle to the hydroformylation step. The process involves removing a crude product stream from a hydroformylation reaction zone comprising one or more aldehyde products, one or more heavies by-products, a transition metal-organophosphorous ligand complex catalyst, one or more unconverted reactants, and one or more inert lights; providing a water stream; combining the water stream with the crude product stream into an azeotropic vaporizer; removing from the vaporizer an overhead gas stream comprising one or more aldehyde products, one or more unconverted reactants, one or more inert lights, a portion of the water, and a portion of the heavies by-products, and feeding said overhead gas stream into a condenser; removing from the condenser an overhead gas stream comprising one or more unconverted reactants, a portion of the added water, and one or more inert lights; removing as a tails stream from the azeotropic vaporizer, a liquid recycle catalyst stream comprising the transition metal-organophosphorous ligand complex catalyst, the balance of the aldehyde product and the balance of the heavies by-products to be sent to the reaction zone wherein at least 25% of the aldehyde product recovered in overhead gas stream from the condenser is removed as a water azeotrope.

### BACKGROUND OF THE INVENTION

This invention pertains to a process of controlling heavies in a catalyst recycle stream. More particularly, this invention pertains to a two-stage process of hydroformylation and product-catalyst separation for controlling heavies in a catalyst recycle stream to the hydroformylation stage.

It is well known in the art that aldehydes can be produced by reacting an olefinically unsaturated compound with carbon monoxide and hydrogen in the presence of a metal-organophosphorus ligand complex catalyst, and that preferred processes involve continuous hydroformylation and recycling of a catalyst solution containing a metalorganophosphorus ligand complex catalyst wherein the metal is selected from Groups 8, 9, or 10. Rhodium is a preferred Group 9 metal. Such art is exemplified by U.S. Pat. Nos. 4,148,830; 4,717,775; and 4,769,498. Aldehydes produced by such processes have a wide range of utility, for example, as intermediates for hydrogenation to aliphatic alcohols, for amination to aliphatic amines, for oxidation to aliphatic acids, and for aldol condensation to produce components of plasticizers.

Commercial hydroformylation of C4 and higher olefins in the presence of a rhodium-triorganophosphine ligand complex catalyst, such as rhodium-triphenylphosphine ligand complex catalyst, is typically conducted in an integrated reaction-separation system similar to that shown in FIG. 1. For example, C4 olefins comprise essentially pure 1-butene or 2-butene streams, as well as mixed C4 raffinate I and raffinate II streams comprising 1-butene, 2-butene, isobutylene, and butane. With reference to FIG. 1, a raffinate stream containing mixed butenes (1) is fed with a stream (2) comprising carbon monoxide and hydrogen (syngas) to a first reactor (Reactor 1). A liquid product stream (3) is removed from the first reactor and fed to a second reactor (Reactor 2), while gas stream (4) taken from the top of the first reactor can also be fed into the second reactor (Reactor 2). Each reactor contains a quantity of rhodium-triphenylphosphine ligand complex catalyst and, optionally, free triphenylphosphine ligand. The complex catalyst and optional free ligand are advantageously solubilized in a liquid heavies by-product comprising aldehyde condensation dimers, trimers, and higher oligomers derived from the hydroformylation of the C4 feed. A gas stream (5) exiting the last reactor can be recycled to the first reactor, or flared, or fed as a fuel to a downstream process. A liquid product stream (6) exiting the last reactor is sent to a vaporizer from which an overhead stream (7) is removed comprising one or more C5 aldehyde product(s), one or more unconverted C4 olefins, unconverted syngas, volatile inerts (e.g., butane), and to some extent heavies by-products. The overhead stream (7) from the vaporizer is condensed at about 40 °C and 10 psig (69 kPa), and the resulting liquid stream (8) is sent to a refining zone (unit not shown) for C5 separation and purification. A vent stream (9) removes volatiles from the condenser. These volatiles comprise mostly nitrogen, carbon monoxide, hydrogen, and less than 1 percent aldehyde products. The vent gases can be flared, routed to a vent recovery stream, or routed to a downstream plant fuel stream. A catalyst recycle stream (10) containing the rhodium-triphenylphosphine ligand complex catalyst and, optional, free triphenylphosphine ligand dissolved in a liquid heavies by-product is obtained from the vaporizer as a liquid tail stream and recycled usually to the first hydroformylation reactor (Reactor 1). The vaporizer operating conditions are adjusted so that the production rate of heavies in the reaction system essentially equals their removal rate in the vaporizer. The vaporizer is operated at about 135 °C and super-atmospheric pressure. Under these vaporizer conditions, the rhodium-triphenylphosphine ligand complex catalyst is thermally stable. Significantly higher temperatures may result in catalyst deactivation. Moreover, the heavies concentration in the catalyst recycle stream to the first reactor usually remains constant, avoiding a build-up of heavies by-products in the recycle stream to the hydroformylation reactor(s).

Present day hydroformylation processes prefer to replace the triorganophosphine ligand with an organophosphite ligand, because the latter possesses higher activity and can produce a higher ratio of normal to branched isomeric aldehyde products. The prior art describes various mono, bis-, and poly-organophosphite ligands for use in modern-day hydroformylation processes. Disadvantageously, organophosphite ligands tend to be less stable as compared with triorganophosphine ligands, that is, more sensitive to thermal degradation. Rhodium-organophosphite catalysts, for example, tend to degrade thermally in the vaporizer at operating conditions suitable for the rhodium-triphenylphosphine ligand. Consequently, it is desirable to operate the vaporizer at a temperature lower than 135 °C in order to minimize thermal degradation of the organophosphite ligand.

Operating the vaporizer at a temperature lower than 135 °C with higher molecular olefins requires the use of sub-atmospheric pressures in order to remove the heavies overhead to the desired extent. The quantity of heavies in the tail stream from the vaporizer should be sufficient to solubilize the catalyst and optional free ligand for recycle in a liquid stream back to the hydroformylation reactors; however, a build-up of heavies in the recycle stream is desirably avoided. Thus, the heavies desirably are removed overhead from the vaporizer at essentially the same rate at which they are formed in the hydroformylation stage, in order to avoid increasing quantities of heavies being returned to the hydroformylation reactors where the heavies would occupy ever increasing reactor volume and reduce productivity. Thus, if the organophosphite catalyst is to be stabilized, and heavies are to be removed to the extent desirable, the vaporizer is required to operate at a temperature lower than 135 °C and at sub-atmospheric pressure. Disadvantageously, condensation of the overhead stream taken from the vaporizer becomes problematic at sub-atmospheric pressure. Condensation temperatures of 0 °C or lower require a costly refrigeration unit and add complexity to the overall system. It would be desirable to avoid this expense and complexity by using a simple water cooling condensation unit for condensing the overhead stream from the vaporizer; but it is not apparent from the prior art how to employ conventional water cooling when desirable organophosphite ligands are employed in the hydroformylation stage.

As the product aldehyde molecular weight increases, the boiling point of the heavies also dramatically increases making their removal more difficult. Higher temperatures in the vaporizer may be needed but these higher temperatures also may also promote the loss of the ligand and/or the formation of more heavies thus a limit is soon reached where it is not possible to vaporize the heavies at or above their rate of formation, limiting catalyst life.

U.S. Pat. No. 6,727,391 discloses a hydroformylation process employing two vaporizers in series, each vaporizer being run at a lower pressure than the hydroformylation reactor and the second vaporizer being run at a lower pressure than the first one. The liquid stream from the second vaporizer is sent to a packed column that is scrubbed with the vapor stream from the second vaporizer. U.S. Pat. No. 6,100,432 discloses a technology similar to U.S. Pat. No. 6,727,391, but only utilizes one vaporizer.

U.S. Pat. No. 6,610,891 discloses a hydroformylation process with minimization of heavies by minimizing the temperature used throughout the reaction and catalyst/product separation zones.

U.S. Pat. No. 5,648,553 discloses several hydroformylation process schemes that use a gas-liquid contact tower immediately after the reactor. A liquid hydroformylation product stream is scrubbed countercurrently with synthesis gas after a vaporization step.

U.S. Pat. No. 5,917,095 discloses a hydroformylation process using a rhodium-organophosphite catalyst with general discussion of catalyst-product separation via vaporization.

U.S. Pat. No. 7,262,330-describes a method for removing water from an hydroformylation system prior to the primary vaporizer to reduce the degradation of phosphite ligand. The goal of this invention is to reduce the presence of water in the vaporizer to as low as possible to avoid ligand hydrolysis.

GB826763A—uses azeotropic distillation of aldehydes in the C4-C6 range with water to separate the normal and the iso aldehyde products downstream of the hydroformylation process. This disclosure is silent on the problem of vaporizing the aldehyde product from the catalyst solution and heavies removal but rather focuses on downstream isomer separation.

U.S. Pat. No. 8,404,903 and US20170355656 disclose stripping gas vaporizers to reduce the distillation conditions while still removing the heavies with minimized catalyst damage. However, the distillation temperatures are still higher than would be preferred to ensure longer catalyst life due heavies buildup especially with higher molecular weight aldehydes. It would be desirable to achieve even lower temperatures and/or higher heavies removal rates to prolong catalyst life especially for hydroformylation processes involving C4 and higher olefins.

WO2021010878A1—This teaches a method for reducing the presence of heavies from the catalyst mixture by using a secondary, non-azeotropic, short residence time vaporizer after a set of primary vaporizers. Although this invention stages the vaporizers, it does not mention azeotropic vaporization on the secondary vaporizer.

EP1232008—This reference utilizes a membrane separation device as a secondary separation unit operation to remove heavies after a primary vaporizer. Similarly, U.S. Pat. No. 10,017,443 has a membrane unit operation as the primary separation step after which the permeate goes to a conventional vaporizer and the retentate is recycled to the reactor. The conventional. non azeotropic vaporizer thermally separates the aldehyde product and the catalyst solution in the vaporizer tails is sent to another membrane unit which removes heavies from the system thus two membrane units are needed.

The rate of heavies formation is a function of a number of variables, but temperature is a key variable. Using the “rule of thumb” of the rate doubling every 10 °C, even a modest decrease in vaporizer temperature may have a dramatic impact on ligand loss and heavies formation rate thus may give significantly longer catalyst life. The presence of water may inhibit the formation of acetal- or ester-based heavies based on the Le Chatelier's Principle as well.

### SUMMARY OF THE INVENTION

In one aspect this invention provides a process of controlling heavies in a catalyst recycle stream, the process comprising:
(a) removing a crude product stream from a hydroformylation reaction zone comprising one or more aldehyde products, one or more heavies by-products, a transition metal-organophosphorous ligand complex catalyst, one or more unconverted reactants, and one or more inert lights
(b) providing a water stream
(c) combining the water stream from step (b) with the stream from step (a) into a vaporizer;
(d) removing from the vaporizer an overhead gas stream comprising one or more aldehyde products, one or more unconverted reactants, one or more inert lights, a portion of the water, and a portion of the heavies by-products, and feeding said overhead gas stream into a condenser;
(e) removing from the condenser an overhead gas stream comprising one or more unconverted reactants, a portion of the added water, and one or more inert lights;
(f) recovering a liquid stream from the condenser comprising the aldehyde product, heavies by-products, and water;
(g) separating the organic components from the water from step (f) in a liquid-liquid separation zone to recover the crude aldehyde product as the top layer and an aqueous phase as the bottom layer; and
(h) removing as a tails stream from the vaporizer, a liquid recycle catalyst stream comprising the transition metal-organophosphorous ligand complex catalyst, the balance of the aldehyde product and the balance of the heavies by-products to be sent to the reaction zone wherein at least 25% of the aldehyde product recovered in step (d) is removed as a water azeotrope.

The process of this invention is advantageously adapted to any step process wherein, firstly, an organophosphorous is employed as a ligand in a transition metal-ligand complex catalyst in a hydroformylation reaction for producing one or more aldehyde products from one or more reactants, and from which, secondly, a crude product stream is obtained and mixed with water then fed into a vaporizer to separate the aldehyde product(s) from the catalyst for recycle of the catalyst back to the first reaction step. For the purposes of this invention, the term “azeotropic vaporizer” is used to describe a vaporizer used to separate a crude product stream from a hydroformylation process fluid stream with the addition of water to the vaporization process such that at least 25% of the resulting aldehyde product was vaporized as the azeotrope. Advantageously, the process of this invention results in a controlled quantity of heavies being recycled to the reaction step, as compared to an increasing quantity of heavies when the process is run under similar conditions with exception that use of added water is not employed. (The comparison assumes that no heavies are deliberately added to the process invention to maintain a higher level of heavies, for example, for solubilization of the catalyst.) Thus, reactor volume remains optimally available for the production of desired product(s) rather than being consumed with ever-increasing volumes of unproductive heavies. The process of this invention is most advantageously adapted to a two-step process wherein an olefin is hydroformylated with carbon monoxide and hydrogen in the presence of a transition metal-organophosphorous ligand complex catalyst, and the resulting crude product mixture is separated in an azeotropic vaporizer to recover the catalyst for recycle to the hydroformylation step.

The advantage of an azeotropic vaporizer is that the vaporization occurs at a much reduced temperature compared to vaporization of the product aldehyde and/or heavies in the absence of the azeotrope. For example, 2-methylpentanal normal boiling point at atmospheric pressure is 118 °C but the water azeotrope boils at 88.5 °C thus a dramatically lower temperature can be employed. Another example is for valeraldehyde which boils at 103 °C and the water azeotrope reduces the boiling point to 83 °C. In addition to aldehyde, heavies can also form azeotropes with water. For example, 2-Ethyl-2 hexenal is an aldol condensation product of butyraldehyde (a “dimer”) which boils at 176 °C and the water azeotrope reduces the boiling point to 97.6 °C.

In a preferred embodiment, the rate of removal of heavies by-products in the overhead gas stream from the azeotropic vaporizer is increased relative to the rate of production of heavies by-products in the hydroformylation step compared to the process without the addition of water.

In a preferred embodiment, the rate of removal of heavies by-products in the overhead gas stream from the vaporizer essentially equals the rate of production of heavies by-products in the hydroformylation step.

### DRAWINGS

FIG. 1 illustrates a conventional integrated process for hydroformylation and separation of a liquid hydroformylation product in a vaporizer, with recycle of a liquid catalyst stream to the hydroformylation.

FIG. 2 illustrates an integrated process of the present invention for hydroformylation and subsequent separation of a liquid hydroformylation product in an azcotropic vaporizer, with recycle of a liquid catalyst stream to the hydroformylation and recycle of water phases.

FIG. 3 illustrates a preferred embodiment of the present invention for hydroformylation and subsequent separation of a liquid hydroformylation product in an azeotropic vaporizer with a preliminary conventional vaporization process prior to the azeotropic vaporizer.

FIG. 4 illustrates a preferred embodiment of the present invention for hydroformylation and subsequent separation of a liquid hydroformylation product in an azeotrope vaporizer with a preliminary membrane catalyst/product separation process prior to the azeotropic vaporizer.

### DETAILED DESCRIPTION OF THE INVENTION

References to the Periodic Table of the Elements herein shall refer to the Periodic Table of the Elements published in Nomenclature of Inorganic Chemistry: IUPAC Recommendations 2005, Royal Society of Chemistry, 2005, ed. N. G. Connelly and T. Damhus. Also, any references to a Group or Groups shall be to the Group or Groups reflected in this Periodic Table of the Elements using the IUPAC system for numbering groups.

All percentages, preferred amounts or measurements, ranges and endpoints thereof herein are inclusive, that is, “less than about 10” includes about 10. “At least” is equivalent to “greater than or equal to,” and “at most” is, thus, equivalent “to less than or equal to.” Numbers herein have no more precision than stated. Thus, “115” includes at least from 114.5 to 115.49. All ranges from a parameter described as “at least,” “greater than,” “greater than or equal to” or similarly, to a parameter described as “at most,” “up to,” “less than,” “less than or equal to” or similarly are preferred ranges regardless of the relative degree of preference indicated for each parameter. Thus, a range that has an advantageous lower limit combined with a most preferred upper limit is preferred for the practice of this invention. The term “advantageous” is used to denote a degree of preference more than required, but less than is denoted by the term “preferably.”

Except in the examples, or where otherwise indicated, all numbers expressing quantities, percentages, properties, functionalities and so forth in the specification are to be understood as being modified in all instances by the term “about.” Unless stated otherwise, when an element, material, or step capable of causing undesirable effects is present in amounts or in a form such that it does not cause the effect to an unacceptable degree it is considered substantially absent for the practice of this invention. Those skilled in the art recognize that acceptable limits vary with equipment, conditions, applications, and other variables but are determinable without undue experimentation in each situation where they are applicable. In some instances, variation or deviation in one parameter is acceptable to achieve another desirable end.

The term “comprising,” is synonymous with “including,” “containing,” or “characterized by,” is inclusive or open-ended and does not exclude additional, unrecited elements, material, or steps. The term “consisting essentially of” indicates that in addition to specified elements, materials, or steps, unrecited elements, materials or steps are optionally present in amounts that do not unacceptably materially affect at least one basic and novel characteristic of the subject matter. The term “consisting of” indicates that only stated elements, materials or steps are present except that unrecited elements, materials or steps are optionally present to an extent that has no appreciable effect, or are substantially absent.

In one aspect this invention provides for a process of controlling heavies in a catalyst recycle stream, the process comprising: (a) removing a crude product stream from a hydroformylation reaction zone comprising one or more aldehyde products, one or more heavies by-products, a transition metal-organophosphorous ligand complex catalyst, one or more unconverted reactants, and one or more inert lights;
(b) providing a water stream;
(c) combining the water stream from step (b) with the stream from step (a) into an azeotropic vaporizer;
(d) removing from the vaporizer an overhead gas stream comprising one or more aldehyde products, one or more unconverted reactants, one or more inert lights, a portion of the water, and a portion of the heavies by-products, and feeding said overhead gas stream into a condenser;
(e) removing from the condenser an overhead gas stream comprising one or more unconverted reactants, a portion of the added water, and one or more inert lights;
(f) recovering a liquid stream from the condenser comprising the aldehyde product, heavies by-products, and water;
(g) separating the organic components from the water from step (f) in a liquid-liquid separation zone to recover the crude aldehyde product as the top layer and an aqueous phase as the bottom layer; and
(h) removing as a tails stream from the vaporizer, a liquid recycle catalyst stream comprising the transition metal-organophosphorous ligand complex catalyst, the balance of the aldehyde product and the balance of the heavies by-products to be sent to the reaction zone wherein at least 25% of the aldehyde product recovered in step (d) is removed as a water azeotrope.

In this invention, we refer hereinafter to a “reactive process”, “reaction fluid”, or “reaction” wherein one or more reactants are contacted in the presence of a metal-organophosphorous ligand complex catalyst, one or more inert lights, and optionally free organophosphorous ligand in an organic solvent to produce a crude liquid product stream comprising one or more reaction products, one or more unconverted reactants, the transition metal-organophosphorous ligand complex catalyst, optionally free organophosphorous ligand, one or more heavies by-products, and one or more inert lights. The preferred process is a hydroformylation process wherein the reactants comprise at least one olefin as described herein as well as carbon monoxide and hydrogen.

The terms “reaction fluid,” “reaction medium” and “catalyst solution” are used interchangeably herein, and may include, but are not limited to, a mixture comprising: (a) a metal-organophosphorous ligand complex catalyst, (b) free organophosphorous ligand, (c) aldehyde product formed in the reaction, (d) unreacted reactants, (e) an organic solvent for said metal-organophosphorous ligand complex catalyst and said free organophosphorous ligand, and, optionally, (f) one or more aldehyde condensation compounds formed in the reaction. The reaction fluid can encompass, but is not limited to, (a) a fluid in a reaction zone, (b) a fluid stream on its way to a at last one separation zone, (c) a fluid in a separation zone, (d) a recycle stream, (c) a fluid withdrawn from a reaction zone or separation zone, (f) a withdrawn fluid being separated in a liquid-liquid separation zone, (g) a separated fluid returned to a reaction zone or separation zone, and (h) a fluid in an external cooler. For the purposes of this invention, the term “heavies” shall refer to liquid by-products of the process characterized as compounds having a normal boiling point 25 degrees Centigrade or more above the normal boiling point of the desired product of the reactive process. In a hydroformylation reaction, for example, the reactant comprises one or more olefins (olefinically unsaturated compounds), the desired product comprises one or more isomeric aldehydes, and the heavies by-products comprise compounds boiling 25 °C or more above the normal boiling point of the aldehyde product. These by-products are typically condensation products of the aldehydes to form esters, alcohols, hemi-acetals, and acetals (and combinations thereof) as described, for example, in U.S. Pat. Nos. 4,148,830 and 4,247,486.

For the purposes of this invention, the term “lights” shall refer to reactants, inerts, by-products of the process, or a combination thereof, characterized as having a normal boiling point at least 25 °C, preferably at least 50 °C, below the normal boiling point of the desired product of the reactive process. As used herein, the term “inert lights” or “light inerts” shall refer to lights that are unreactive in the process. “Reactive lights” shall refer to lights that are reactive in the process. As an example, in a hydroformylation process, reactive lights include carbon monoxide and hydrogen; while inert lights include alkanes present in the olefinic feed to the reaction.

In another aspect, the process further comprises passing the crude product stream from step (a) to a preliminary product/catalyst separation process prior to step (c). This preliminary product/catalyst separation process may advantageously involve a distillation or vaporization process wherein the top stream is a first crude aldehyde product stream and the bottoms is a transition metal-organophosphorous ligand complex catalyst and heavies stream a portion of which is then fed to step (c). Alternatively this preliminary product/catalyst separation process may involve a membrane separation process wherein the retentate comprises a transition metal-organophosphorous ligand complex catalyst, some aldehyde product and heavies stream which is recycled to the reaction zone and the permeate comprises aldehyde product, at least some heavies, unconverted reactants, inert lights and some catalyst components which is then fed to step (c). Alternatively, a liquid-liquid phase separation process may be employed as a preliminary product/catalyst separation process such as described in U.S. Pat. No. 5,952,530 wherein the product phase (containing traces of catalyst) and/or the catalyst-containing phase (containing substantial product aldehyde) can be fed to one or more azeotropic vaporizers specific for that particular stream as step (c) described above. Preferably, a majority of the total aldehyde product produced in the hydroformylation reaction zone is separated in the preliminary product/catalyst separation zone.

In another aspect of the invention, at least a portion of the bottom water phase from step (g) is recycled to step (b).

In another embodiment of the present invention, a buffer to maintain suitable pH values is added to the water stream prior to it being fed into the vaporizer.

In another aspect of the invention, the water stream of step (b) is in the form of steam.

In some embodiments the azeotropic vaporizer is a stripping gas vaporizer.

In a preferred embodiment, the rate of removal of heavies by-products in the overhead gas stream from the azeotropic vaporizer essentially equals the rate of production of heavies by-products in the hydroformylation reactor. In a preferred embodiment, the rate of removal of heavies by-products in the overhead gas stream from the azeotropic vaporizer can exceed the rate of production of heavies by-products in the hydroformylation reactor.

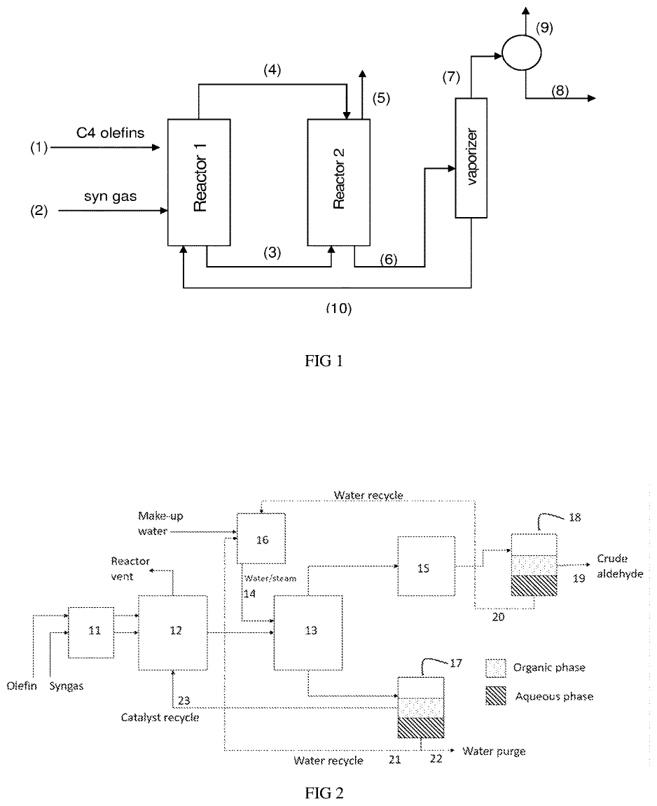

With reference to FIG. 2, which embodies the invention as it pertains to a hydroformylation process with subsequent product-catalyst separation, olefin and syngas feedstock are fed into a feedstock pretreatment block (11) following which the feedstock streams are fed into reaction zone (12). For purposes of the present invention, the olefin can range from C3-C20 provided that the aldehyde synthesized from it forms a minimum-boiling azeotrope with water. The determination of whether the aldehyde forms a minimum-boiling azeotrope can be done by methods well known in the art. Reaction zone (12) can be a single reactor or a collection of reactors in series or in parallel and can range from stirred tank reactors, jet mixed reactors, plug flow reactors, bubble column reactors, or a mixture thereof.

The aldehyde product formed in reaction zone (12) along with the catalyst solution is fed into an azeotropic vaporizer (13). Liquid water or steam is also introduced into the azcotropic vaporizer (13) through the water reservoir (16) via line (14). The organic phase from the reaction zone (12) and the aqueous phase from line (14) can be pre-mixed prior to entering the azeotropic vaporizer (13) through the use of static in-line mixers. Alternatively, the design of the azeotropic vaporizer could include provisions for intimate contact between the two phases as they get vaporized before or when entering the vaporizer. The amount of water/steam added to the system depends on the aldehyde being produced, the catalyst system and the concentration of the solvent, if used. The catalyst used in hydroformylation, whether as the Rh-ligand complex or the ligand itself, is chosen such that they do not form an azeotrope with water. The amount of water entering the azeotropic vaporizer (13) will have a higher concentration than the solubility limit of water in the catalyst solution/aldehyde product that exits the reaction zone (12). Thus, a liquid-liquid contactor between water and the catalyst solution to feed water into the catalyst solution will be insufficient and cannot be used as the sole source of water (such a system will not be able to exceed the solubility limit of water in the catalyst solution). The azeotropic vaporizer, under the right operating conditions as taught herein, vaporizes the water-oxygenated product azeotrope which is condensed in condenser (15). The oxygenated product is majorly the aldehyde product but can also include heavier hydroformylation by-products dubbed as heavies that may form an azeotrope with water. The azeotropic vaporizer (13) can be configured to run where the liquid phases and the gas phase flow concurrently, e.g., a rising film evaporator, or counter-currently with respect to each other, e.g., a falling film evaporator. A provision can also be considered where the azeotropic vaporizer is operated with a strip gas loop (“stripping gas vaporizer”) as disclosed in U.S. Pat. No. 8,404,903 and US20170355656. Another provision can be considered where the azeotropic vaporizer is a distillation column.

Upon condensing the vapor in condenser (15), the two liquid phases, viz. organic phase, and the aqueous phase, are obtained and are transferred to the liquid-liquid (L-L) separator (18). The condenser and L-L separator may be one unit in some embodiments. The bottoms of the azeotropic vaporizer which contains the catalyst solution is preferably fed into the L-L separator (17) to separate out any liquid water from the catalyst solution, before the catalyst solution is recycled back into reaction zone (12) via line (23). Optionally, an extractor may be present in the catalyst recycle stream, as described in U.S. Pat. No. 10,131,608, either after L-L separator (17) (if used) or between the vaporizer and the reaction zone.

Due to the formation of two nearly immiscible phases in both L-L separators (organic and aqueous), a physical separation is possible which simplifies the separation of the components that form an azeotrope. Upon separation, the crude aldehyde product is obtained from L-L separator (18) via line (19).

The aqueous phases from L-L separator (17) and/or (18) can be recycled back into the water reservoir (16) via lines (21) and (20), respectively, to reduce the water usage. An optional water purge stream (22) from L-L separator (17) may be used to purge any unwanted species from building up in the aqueous phase. A water make-up feed may be added to the water reservoir (16) to account for any water lost from the system.

Olefinically-unsaturated compounds suitably employed in the process of this invention are those that are capable of participating in a hydroformylation process to produce corresponding aldehyde product(s) and capable of being separated from the crude liquid hydroformylation product stream via vaporization and subsequent decantation. For the purposes of this invention, an “olefin” is defined as an aliphatic organic compound containing at least carbon and hydrogen atoms and having at least one carbon-carbon double bond (C═C). Preferably, the olefin contains one or two carbon-carbon double bonds, more preferably, one carbon-carbon double bond. The double bond(s) can be located at a terminal position along the carbon chain (alpha olefin) or at any internal position along the chain (internal olefin). Optionally, the olefin can comprise elements other than carbon and hydrogen including, for example, nitrogen, oxygen, and halogens, preferably, chlorine and bromine. The olefin can also be substituted with functional substituents including, for example, hydroxy, alkoxy, and alkyl substituents. Preferably, the olefin used in the process of this invention comprises a substituted or unsubstituted olefin having a total of from 3 to 20 carbon atoms and most preferably between 4-18 carbons. Illustrative olefins suitable for the process of this invention include, without limitation, propylene and isomers of the following monoolefins including butene, pentene, hexene, heptene, octene, nonene and decene, with specific non-limiting examples including 1-butene, 2-butene, 1-pentene, 2-pentene, and 1-hexene, 2-hexene, 3-hexene, and similarly, for heptene, octene, nonene, and decene. Other non-limiting examples of suitable olefins include 2-methyl propene (isobutylene), 2-methylbutene, cyclohexene, isoprene, 2-ethyl-1-hexene, styrene, 4-methyl styrene, 4-isopropyl styrene, 4-tert-butyl styrene, alpha-methyl styrene, 3-phenyl-1-propene, 1,4-hexadiene, and 1,7-octadiene.

Preferably, the olefin stream used in the process of this invention comprises a C4 raffinate I or C4 raffinate II isomeric mixture comprising butene-1, butene-2, isobutylene, butane, and optionally, butadiene. The C4 raffinate I stream comprises from 15 to 50 percent isobutylene and from 40 to 85 percent normal butenes, by weight, any remainder to 100 percent comprising primarily n-butane and isobutane. The normal butenes are generally a mixture of butene-1 and butene-2 (cis- and trans-forms). The relative proportions stream components depend upon the composition of the petroleum feed, the conditions employed in steam cracking or catalytic cracking operation, and in the subsequent process steps, from which the C4 stream is derived. The C4 raffinate II stream comprises from about 15 to 55 percent 1-butene, from about 5 to about 15 percent 2-butene (5 to 35 percent trans-2-butene), from about 0.5 to about 5 percent isobutylene, and from about 1 to about 40 percent butane, by volume.

Other embodiments include higher molecular weight olefin starting materials such as C5-C20 olefins as defined herein. For example, in embodiments, when the olefin starting materials are mixed C8 olefins, the mixed C8 olefins include mixtures such as may be obtained via dimerization of mixed butenes comprising 1-butene, cis and trans-2-butene and optionally isobutene. In one embodiment, a stream comprising mixed octenes derived from the dimerization of Raffinate II is employed; such mixtures may be produced, for example by the Dimersol process from Axens (Institut Français du Petrole, Review, Vol. 37, No 5, September-October 1982, p 639) or the Octol process from Hüls A G (Hydrocarbon Processing, February 1992, p 45-46). It is understood that the olefin mixtures employed in the process of the invention may also comprise some amount of linear alpha olefins.

In some embodiments, the olefin starting materials are mixed C9 olefins as defined herein. Such mixtures may be available from a variety of sources and may be produced, for example by the process described in Johan A. Martens, Wim H. Verrelst, Georges M. Mathys, Stephen H. Brown, Pierre A. Jacobs “Tailored Catalytic Propene Trimerization over Acidic Zeolites with Tubular Pores”, Angewandte Chemie International Edition Angewandte Chemie International Edition 2005, Volume 44, Issue 35, pages 5687-5690.

It should be understood that embodiments of the present invention are designed for use in processes where the olefin starting materials are C3-20 olefins such as described above. In some embodiments, the olefin starting materials are primarily mixed C8 olefins or mixed C9 olefins and may include either mixed C8 olefins or mixed C9 olefins. However, it should be also understood that in processes designed for the hydroformylation of mixed C8 olefins, a small amount of mixed C9 olefins may also be present in the olefin starting materials. Likewise, it should be also understood that in processes designed for the hydroformylation of mixed C9 olefins, a small amount of mixed C8 olefins may also be present in the olefin starting materials.

Hydrogen and carbon monoxide are also required for the hydroformylation step of this invention. These gases can be obtained from any available source including petroleum cracking and refinery operations. Synthesis gas mixtures are preferably employed. The H₂:CO molar ratio of gaseous hydrogen to carbon monoxide can range, preferably, from about 1:10 to about 100:1, the more preferred H₂:CO molar ratio being from about 1:10 to about 10:1, and even more preferably, from about 1:10 to about 1:2.

Transition metal-ligand complex catalysts employable in the hydroformylation process of this invention, as well as methods for their preparation, are well known in the art. In general, such catalysts can be preformed or formed in situ and consist essentially of a transition metal in complex combination with an organophosphorus ligand, preferably, an organophosphite ligand. Suitable transition metals which make up the metal-ligand complexes include Group 8, 9 and 10 metals selected from rhodium (Rh), cobalt (Co), iridium (Ir), ruthenium (Ru), iron (Fe), nickel (Ni), palladium (Pd), platinum (Pt), osmium (Os) and mixtures thereof, with the preferred metals being rhodium, cobalt, iridium and ruthenium, more preferably rhodium, cobalt and ruthenium, and most preferably, rhodium. Other suitable metals include Group 6 metals selected from chromium (Cr), molybdenum (Mo), tungsten (W) and mixtures thereof. Mixtures of metals from Groups 6, 8, 9 and 10 can also be used in this invention.

Preferred organophosphorous ligands that make up the metal-organophosphorous ligand complex and free organophosphorous ligand include mono-, di-, tri- and higher organophosphites, as described in detail in U.S. Pat. No. 8,404,903. Mixtures of such ligands can be employed if desired in the metal-organophosphite ligand complex catalyst and/or free ligand, and such mixtures can be the same or different.

A most preferred organophosphorous ligand is a triorganophosphite ligand such as tris-(2,4-di-tert-butylphenyl)phosphite represented by Formula W:

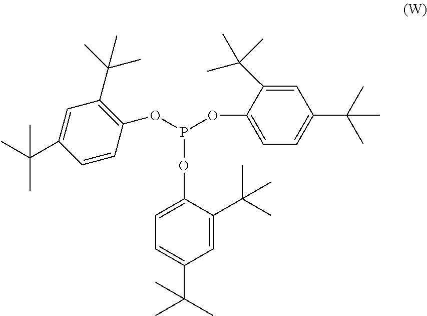 [SMILES: CC(C)(C)c1ccc(OP(Oc2ccc(C(C)(C)C)cc2C(C)(C)C)Oc2ccc(C(C)(C)C)cc2C(C)(C)C)c(C(C)(C)C)c1]

A second preferred triorganophosphite ligand comprises (tris(2-t-butyl-4-methylphenyl)phosphite represented by Formula X:

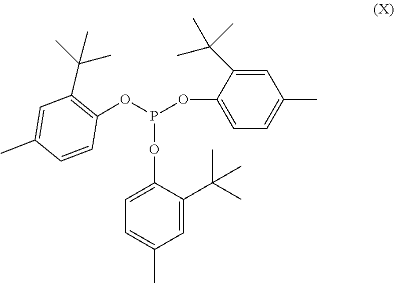

In a preferred embodiment of this invention, the organophosphorous ligand comprises an organobisphosphite ligand.

Another class of preferred organophosphorous ligands that make up the metal-organophosphorous ligand complex and free organophosphorous ligand include non-ionic triarylphosphines, as described in detail in U.S. Pat. No. 3,527,809. Mixtures of such triarylphosphine ligands can be employed if desired in the metal-organophosphorous ligand complex catalyst and/or free ligand. A preferred triarylphosphine is triphenylphosphine.

The term “complex” as used herein and in the claims means a coordination compound formed by the union of one or more electronically rich molecules or atoms with one or more electronically poor molecules or atoms. For example, the organophosphorous ligands employable herein possess one or more phosphorus donor atoms, each having one available or unshared pair of electrons, each pair of which is capable of forming a coordinate covalent bond independently or in concert (for example, via chelation) with the metal. Carbon monoxide can also be present and complexed with the metal. The complex catalyst can also contain an additional ligand, for example, hydrogen or an anion satisfying the coordination sites or nuclear charge of the metal. Illustrative additional ligands include, for example, halogen (Cl, Br, I), alkyl, aryl, substituted aryl, acyl, CF₃, C₂F₅, CN, (R)₂PO and RP(O)(OH)O (wherein each R is the same or different and is a substituted or unsubstituted hydrocarbon radical, for example, alkyl or aryl), acetate, acetylacetonate, SO₄, PF₄, PF₆, NO₂, NO₃, CH₃O, CH₂═CHCH₂, CH₃CH═CHCH₂, C₂H₅CN, CH₃CN, NH₃, pyridine, (C₂H₅)₃N, mono-olefins, diolefins and triolefins, tetrahydrofuran, and the like.

The number of available coordination sites on the aforementioned transition metals is well known in the art. Thus, the catalytic species can comprise a complex catalyst mixture in monomeric, dimeric and/or higher nuclearity forms, which are preferably characterized by at least one organophosphorus-containing molecule complexed per one molecule of transition metal, for example, rhodium. The catalytic species of the preferred catalyst employed in the hydroformylation reaction can be complexed with carbon monoxide and hydrogen in addition to the organophosphorous ligand(s) in view of the carbon monoxide and hydrogen gas employed by the hydroformylation reaction.

The amount of transition metal-ligand complex catalyst present in the hydroformylation step is that minimum amount necessary to provide a metal concentration necessary to catalyze the selected hydroformylation process. In general, a metal concentration, for example, rhodium concentration, in a range from about 10 parts per million to about 1000 parts per million, calculated as free metal in the hydroformylation reaction fluid is sufficient for most processes; while it is generally preferred to employ from about 10 to 500 parts per million of metal, and more preferably from 25 to 350 parts per million of metal.

Optionally, free ligand (that is, ligand that is not complexed to metal) can also be present in the hydroformylation reaction fluid. The free ligand can correspond to any of the aforementioned organophosphorous ligands. The hydroformylation process of this invention can involve advantageously from about 0.1 to about 300 moles of free organophosphorous ligand per mole of metal in the hydroformylation reaction fluid. Preferably the hydroformylation is conducted in the presence of from about 1 to about 50 moles of organophosphite ligand, and more preferably from about 1.1 to about 4 moles of organophosphite ligand, per mole of metal present in the reaction fluid; said amounts of ligand being the sum of both the amount of bound ligand complexed to the metal present and the amount of free (non-complexed) ligand present. With triarylphosphines, the organophosphorous to rhodium ratio is typically much higher, typically comprising 5-20 wt % of the reaction fluid which typically corresponds to 30 to 300 moles of triarylphosphine to moles catalytic metal. If desired, make-up or additional ligand can be supplied to the hydroformylation process at any time and in any suitable manner, for example to maintain a predetermined level of free ligand in the reaction fluid.

The reaction conditions of the hydroformylation process can vary widely. For instance, the H₂:CO molar ratio of gaseous hydrogen to carbon monoxide advantageously can range from about 1:10 to 100:1 or higher, the more preferred hydrogen to carbon monoxide molar ratio being from about 1:10 to about 10:1. Advantageously, the hydroformylation process can be conducted at a reaction temperature greater than about −25 °C, more preferably, greater than about 50 °C. The hydroformylation process advantageously can be conducted at a reaction temperature less than about 200 °C, preferably, less than about 120 °C. Advantageously, the total gas pressure comprising olefinic reactant, carbon monoxide, hydrogen, and any inert lights can range from about 1 psia (6.8 kPa) to about 10,000 psia (68.9 MPa). Preferably, the process be operated at a total gas pressure comprising olefinic reactant, carbon monoxide, and hydrogen of less than about 2,000 psia (6,895 kPa), and more preferably, less than about 500 psia (34.5 kPa). Advantageously, the carbon monoxide partial pressure varies from about 1 psia (6.8 kPa) to about 1000 psia (6,800 kPa), and preferably from about 3 psia (20.7 kPa) to about 800 psia (5,516 kPa), and more preferably, from about 15 psia (103.4 kPa) to about 100 psia (689 kPa); while the hydrogen partial pressure varies preferably from about 5 psia (34.5 kPa) to about 500 psia (3,450 kPa), and more preferably from about 10 psia (68.0 kPa) to about 300 psia (2,070 kPa).

The feed flow rate of synthesis gas (CO+H₂) can vary widely over any operable flow rate sufficient to obtain the desired hydroformylation process. The syngas feed flow rate depends upon the specific form of catalyst, olefin feed flow rate, and other operating conditions. Likewise, the vent flow rate from the Oxo reactor(s) can be any operable flow rate sufficient to obtain the desired hydroformylation process. Vent flow rate is dependent upon the scale of the reactor and the purity of the reactant and syngas feeds. Suitable syngas feed flow rates and vent flow rates are described in the following reference: “Process Economics Program Report 21D: Oxo Alcohols 21d,” SRI Consulting, Menlo Park, California, Published December 1999, incorporated herein by reference. Other syngas and vent flow rates can be suitable depending upon the design of the process, as determined by one skilled in the art.

The crude liquid output from the hydroformylation reactor system can be fed directly into the azeotropic vaporizer. If desired, the crude liquid output from the hydroformylation reactor system can be fed first into a flash column or “knock-out pot” to let down pressure and remove a small vent stream of volatiles of low molecular weight (lights), such as carbon monoxide, hydrogen, N₂, and other inert lights; after which the remaining bulk liquid product is removed from the bottom of the flash column and fed to the azeotropic vaporizer.

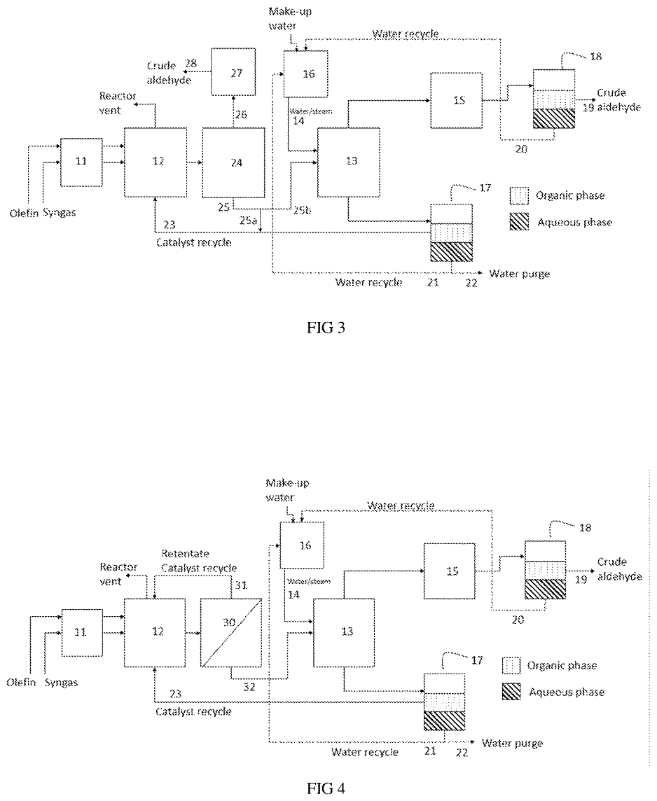

In a preferred embodiment, the crude liquid output from the hydroformylation reaction system or the flash column can be fed to a preliminary catalyst/product separation process to either remove some of the product or some of the catalyst prior to feeding to the azeotropic vaporizer. For example, in a preferred embodiment as shown in FIG. 3, a conventional vaporizer (24) (non-azeotropic) is used first to remove as much crude aldehyde product as possible via line (28) before at least a portion of the preliminary vaporizer tails is sent via line (25b) to the azeotropic vaporizer (13). This concentrates the heavies, thus the heavies azeotrope is enhanced. The preliminary vaporizer (24) is a traditional vaporizer or a stripping gas vaporizer that uses thermal energy to vaporize the product aldehyde and retain the catalyst solution in liquid form. The vaporized aldehyde is condensed in a condenser (27) which, optionally, yields the majority of the total crude aldchyde produced via line (28). Preferably, this is the primary product removal process for the system. A provision can be made where only a slip stream (25b) of the preliminary vaporizer (24) tails can be fed into the secondary azeotropic vaporizer (13) and the remainder recycled back to the reaction zone via lines (25a) and (23). This slip stream option reduces the amount of water and steam needed to run the azeotropic vaporizer, reduces the size of the azeotropic vaporizer, and reduces any stresses on the total ligand inventory due to the stripping conditions in the azeotropic distillation to remove the heavies. As described herein, the conventional preliminary vaporizer (24) and the azeotropic vaporizer (13) can be configured to run where the liquid phases and the gas phase flow concurrently, e.g., a rising film evaporator, or counter-currently with respect to each other, e.g., a falling film evaporator or in different modes. The crude product from the azcotropic vaporizer (via (19)) can be combined with the crude product from the preliminary vaporizer (28) or processed separately.

Alternatively, the crude liquid output from the hydroformylation reactor system can be fed to an intermediate product-catalyst separation process to remove and recycle a portion of the catalyst prior to feeding the resulting material to the azeotropic vaporizer (13). Examples of such an intermediate product-catalyst separation processes include a membrane separation unit such as described in U.S. Pat. No. 10,017,443. This preferred scheme is shown in FIG. 4 where the primary mode of catalyst and product separation is a membrane unit instead of a vaporizer. In FIG. 4, this is depicted by membrane unit (30) and the other unit operations remain the same. Here the membrane unit (30) selectively splits the organic stream from reaction zone (12) into a retentate stream (31) and a permeate stream (32) based on the properties of the membrane. Generally, membranes are selective towards retaining the catalyst solution in the retentate and pushing the heavies into the permeate. However, in most cases, some amount of both the catalyst and the heavies are in the permeate and the retentate respectively. Thus, the catalyst rich retentate (31) can be fed back to the reactor and the product, heavies and part of the catalyst containing permeate (32) can be fed to the azeotropic vaporizer (13). Here the azeotropic vaporizer is a secondary separation unit operation, performing the same function as described above with respect to FIG. 2.

Benefits of using a preliminary product/catalyst separation process followed by a secondary azcotropic vaporizer include:
(1) Feed to tails ratio (F/T), the ratio of the stream entering the vaporization system compared to the catalyst recycle stream to the reaction zone) can be increased as compared to a one vaporizer system. With just one traditional vaporizer, the amount of aldehyde and heavies vaporized is limited by the vaporizer temperature. The upper limit of this temperature is determined by the catalyst degradation temperature. This limits the F/T ratio or the recycle ratio which reduces the overall productivity of the system. With a secondary azeotropic vaporizer, additional aldehyde can be vaporized with the aid of water (which forms the azeotrope). Therefore, the productivity of the overall plant can be increased by vaporizing additional aldehyde via increasing the overall F/T ratio without increasing the severity of operation. A higher F/T ratio generally results in longer residence time in the reactors thus results in higher olefin conversions.
(2) This system will have a lower water requirement and lower energy requirement as compared to just using azeotropic vaporizer as the primary vaporizer. Since the amount of water (and steam needed to vaporize that water) is higher to generate the azeotrope for higher olefins, this scheme is more suited for higher molecular weight olefins as it reduces the water amount required. Generally, the higher the molecular weight of the oxygenated compound, the larger is the amount of water required to form the azeotrope.
(3) The removal of heavies in the azeotropic vaporizer may be sufficient to eliminate/reduce a heavies purge stream and eliminate/reduce the duty of the subsequent catalyst recovery process from the purge stream. When the organic feed to the azeotropic vaporizer is from the preliminary separation unit operation, this stream contains a higher concentration of heavies as the aldehyde has either been evaporated in the preliminary vaporizer or the heavies have been selectively separated in a membrane separator. A higher concentration of heavies tends to form an azeotrope with water which has a higher selectivity towards vaporizing heavies as compared to a lower concentration heavies stream. Aldehyde is still the major product which is vaporized from the azeotropic vaporizer.
(4) Reduces the severity of the primary vaporizer: With the secondary azeotropic vaporizer vaporizing part of the aldehyde in addition to vaporizing heavies, the primary vaporizer can run at milder conditions which can help with longer catalyst life and lower ligand degradation. By decoupling product and heavies removal processes, each process can be optimized to their particular task rather than a compromise between them if only a single product/catalyst separation process is used.

The feed to the azeotropic vaporizer comprises an organic phase from the hydroformylation reaction zone (12) described above and an aqueous phase (14) as shown in FIG. 2. The water to be added to form the azeotrope in the azeotropic vaporizer can be added to the crude liquid output from the hydroformylation reactor system prior to being fed to the azeotropic vaporizer or as a separate stream or both. The water from the water reservoir (16) can be pre-mixed with the organic phase prior to entering the azeotropic vaporizer (13) through the use of static in-line mixers or actively mixed. In the former case, the two stream should be mixed prior to entering the reactor as much as possible although the mixture will not be homogenous. The mixing will ensure a more even distribution of the two phases into the azeotropic vaporizer which will result in a more stable system. In this scenario, the water is preferably added as a liquid to the organic liquid phase, preferably pre-heated to at or between the reaction zone temperature and the azeotropic vaporizer temperature.

Alternatively, the design of the azeotropic vaporizer could include provisions for intimate contact between the two phases as they get vaporized before or when entering the azeotropic vaporizer (13). In the case where the water is introduced separately from the crude liquid output, the water can be as liquid water or steam.

The water reservoir (16) in FIG. 2 may be a tank or other suitable device to supply water to the system and may incorporate heating and de-aerating capabilities. The reservoir may also allow for optional additives to be added and mixed prior to the water being introduced to the azeotropic vaporizer (13). Such additives include but are not limited to weakly basic amines such as described in U.S. Pat. No. 10,131,608.

The amount of water/steam added to the system depends on the aldehyde being produced, the catalyst system and the concentration of the solvent, if used. The amount of water entering the azeotropic vaporizer (13) will be higher than the solubility limit of water in the catalyst solution/aldehyde product that exits the reaction zone (12). The azeotropic vaporizer (13), under the operating conditions as taught herein, vaporizes the water-oxygenated product azeotrope which is condensed in condenser (15).

The amount of water to be added is that needed to maintain the azcotropic distillation in the azeotropic vaporizer. Insufficient water will result in lower productivity and higher temperatures and too much water will result in possibly higher steam usage and higher water flow in the optional L-L separator (17). There are two convenient means to determine the optimal amount of water to be added at step (c):
1. Compare the amount of water and organic phases obtained in step (g) to the amount of water and organic phase predicted from off-line experiments which indicate what the azeotrope values should be and adjust the amount of water being added to match the predicted values.
2. A properly running azeotropic vaporizer running at a high azeotropic distillation level (above 90%) should have at least some water remaining at the bottom in the azcotropic vaporizer tails thus measuring the amount of water present at the bottom of the azeotropic vaporizer (e.g., the tails stream from step (i)) should have at least some water present to insure the entire azeotropic vaporizer was run under azeotropic conditions. If L-L separator (17) is not used, the water level should be at least the saturation limit at the azeotropic vaporizer bottoms temperature (typically 0.1 wt %) but can be higher if the L-L separator is used to minimize liquid water being transferred to the reaction zone.

The composition of the azeotrope used in the first method for controlling the amount of water to be added can be determined by means well known in the art and exemplified in our examples. Based on the flows of the organic (19) and aqueous phases (20) while maintaining a constant total liquid level and a constant water/organic interface level in the L-L separator (18), the relative amounts of organic phase and aqueous phase can be determined and compared to the ratio predicted by off-line experiments such as shown in our examples. Excessive water flow in stream (21) (if L-L separator (17) is used) is also indicative of too much water being added.

Likewise, the measurement of the water content of the liquid recycle catalyst stream from the bottom of the azeotropic vaporizer (13) can be done by conventional means such as Karl-Fischer analysis or on-line methods such as infra-red or near infra-red (IR) analysis techniques including Fourier transform IR (FTIR) and Raman spectroscopy.

Regardless of the methods used to determine the amount of water to be added and the conditions in the azeotropic vaporizer, the amount of water being added back to the reaction zone in the catalyst recycle stream (23) should be low enough as to not form a significant separate water phase in the reactor. This is preferably done using L-L separator (17). A separate water phase under the reaction conditions in the reaction zone (12) represents non-productive volume since the catalyst and olefin are not soluble in the water phase. The concentration of water that is soluble in the catalyst solution at the reaction zone temperatures can be readily determined by conventional means and the presence of an aqueous phase should be less than 1 wt % of the total liquid phase in the reaction zone.

The azeotropic vaporizer (13) is conventional in design as known to the skilled person. Azcotropic vaporizers are advantageously designed as a vertical, tubular heat exchanger with a heating means. The azeotropic vaporizer dimensions (number of tubes, diameter and length) are determined by the plant capacity and are limited only by the vendor's fabrication shop capabilities. There are usually no internals other than a liquid and gas distributor that is built into the inlet head of the heat exchanger to insure good distribution of the feeds. The crude liquid product stream, comprising one or more products, one or more heavies by-products, a transition metal-organophosphorous ligand complex catalyst, one or more unconverted reactants, one or more reactive lights, and optionally, one or more inert lights, is advantageously fed into the top ⅓, preferably, top head of the azeotropic vaporizer at a temperature and pressure appropriate for obtention of the desired overhead gas stream comprising a portion of the heavies by-products and liquid recycle tail stream comprising the balance of the heavies by-products and the transition metal-organophosphorous ligand complex catalyst. In the preferred embodiment of this invention, wherein the input is a liquid hydroformylation product stream comprising one or more aldehyde products, one or more heavies by-products, one or more unconverted olefinic reactants, a transition metal-organophosphorous ligand complex catalyst, optionally free organophosphorous ligand, carbon monoxide, hydrogen, and inert lights, the azeotropic vaporizer is operated at a temperature sufficiently high enough to remove at least a portion of the heavies in the gas overhead stream while sufficiently low enough to ensure stability of the catalyst and organophosphorous ligand in the azcotropic vaporizer. Preferably, the azcotropic vaporizer temperature is greater than about 80 °C, more preferably, greater than about 90 °C. Preferably, the azeotropic vaporizer temperature is less than about 130 °C, more preferably, less than about 120 °C. The azeotropic vaporizer pressure advantageously is greater than about 14 psia (96.5 kPa), preferably, greater than about 20 psia (138 kPa). The azeotropic vaporizer pressure is advantageously less than about 100 psia (689 kPa), preferably, less than about 60 psia (414 kPa). The azeotropic vaporizer operates advantageously with a mass ratio of crude liquid product feed to liquid tails ranging from about 2/1 to about 5/1, preferably, from about 2.0/1 to about 3.0/1.

Preferably a stripping gas vaporizer is used as described in U.S. Pat. No. 8,404,903 U.S. 20,170,355656 for the azeotropic vaporizer. The mass ratio of crude liquid product feed to recycle gas feed to the vaporizer is preferably greater than about 0.1/1, more preferably, greater than about 0.5/1, but preferably, less than 2/1, and more preferably, less than about 1/1.

The overhead gas stream from the azeotropic vaporizer is fed into a condenser (15). The condenser advantageously employs conventional water cooling; no special refrigeration unit is required. Water is the preferred cooling liquid at an operating temperature ranging from above freezing (i.e., greater than 10 °C) to about 50 °C, preferably, from about 34 °C to about 45 °C. The condensed liquid is sent to a liquid-liquid separator (18) and any uncondensed gases are either vented (not shown in FIG. 2) or recycled to the azeotropic vaporizer as part of the stripping gas as described in U.S. Pat. No. 8,404,903 and US20170355656 (not shown in FIG. 2).

The condensed liquid from the condenser is separated into an organic layer and an aqueous layer in a liquid-liquid separation zone (18). The design of such a separation device is not narrowly critical to this invention and is well known to those skilled in the art and are typically operated at or below the cooling temperature of the condenser and at or below the pressure of the condenser. The organic phase comprising the product aldehyde, heavies, and traces of water and other materials are withdrawn via line (19) for further processing (e.g., refining, hydrogenation, aldol condensation). Given the high molecular weight of the aldehyde product and heavies, the solubility of water in the organic phase is typically quite low thus very little water is lost in the crude aldehyde product stream. This water may be recovered in downstream processing and recycled if desired. The aqueous layer is preferably recycled back to the azcotropic vaporizer via line (20) to unit (16) as part of the water introduced to the azeotropic vaporizer for the azeotropic distillation. A purge on line (20) may also be present (not shown in FIG. 2).

In one preferred embodiment, the aqueous stream from the bottom of unit (18) is sent through a cross heat exchanger enroute to unit (16) as part of the condensation process in unit (15) to maximize the heat integration within the plant and minimize steam costs.

The liquid top layer stream from the liquid-liquid separator (18) comprises predominantly one or more aldehyde products, a portion of the unconverted olefinic reactants, water, a portion of the inert lights, and a portion of the heavies by-products. Advantageously, the top liquid stream (19) comprises from about 10 to about 95 percent aldehyde products, from about 8 to about 28 percent unconverted olefinic reactants, from about 6 to about 26 percent inert lights, predominantly, inert alkanes, 0.1 to 10 wt % water, and from about 0.01 to about 0.2 percent heavies by-products, by weight.

Although the heavies by-products leaving the condenser from tails stream from (15) in stream (19) at any given unit of time comprise a small fraction of the liquid stream from the condenser, this heavies output is responsible for reducing the build-up of heavies by-products in the hydroformylation step. Preferably, the fraction of heavies in (19) per unit time is essentially equivalent to the fraction of heavies by-products produced per identical unit of time in the hydroformylation process. In this instance, the heavies are removed from the reaction system essentially at the same rate at which they are being produced. Thus, there is no undesirable increase in heavies recycled to the hydroformylation step; and heavies recycled to the hydroformylation step can remain in essentially a steady state at just the desired quantity necessary to solubilize the catalyst. The reduction in the azeotropic vaporizer temperature enabled by employing the water azeotrope will enhance the removal of heavies as well as possibly reducing the loss of ligand and formation of additional heavies in the azeotropic vaporizer (13).

The partial pressure of carbon monoxide in the overhead gas of the azeotropic vaporizer and condenser can vary dramatically depending on the azeotropic vaporizer conditions as discussed in U.S. Pat. No. 8,404,903 and US20170355656. The partial pressure of carbon monoxide, hydrogen, or other inert gases are not narrowly critical for the present invention but will contribute to the system total pressure which is typically controlled by a vent on the condenser (not shown in FIG. 2) and/or as part of the recycle loop as taught in U.S. Pat. No. 8,404,903 and US20170355656.

Referring to FIG. 2, the liquid tails stream from (13) obtained from the azeotropic vaporizer comprises predominantly heavies and the transition metal-organophosphorous ligand complex catalyst and can further comprise one or more aldehyde products and/or free organophosphorous ligand. Generally, this liquid tails stream, which we refer to as the liquid catalyst recycle stream, comprises from about 40 to about 88 percent heavies by-products and from about 7 to about 27 percent aldehyde product(s), by weight, exclusive of the weight of the transition metal-organophosphorous ligand complex catalyst, any optional free organophosphorous ligand, and possibly water. The stream can also comprise small quantities of unconverted olefinic reactants and inert alkanes. Clearly, the complex catalyst and ligand are not volatile, and therefore, essentially all of the catalyst and ligand are recycled to the hydroformylation reactor in liquid catalyst recycle stream. The aqueous stream (21) consists of water from about 85-99.99% and organics from the organic stream from about 0.01-15%. Overall the aqueous to organic stream split ((21)+ (22))/(23) ratio) can be from about 0.5 to 100

Typically the liquid tail stream from the azeotropic vaporizer is cooled prior to being sent back to the reaction zone in part to reduce ligand degradation and heavies formation during that transit. In some embodiments, the cooled azeotropic vaporizer tails stream is sent to a liquid-liquid separator shown in FIG. 2 as unit (17). Any liquid water is separated and preferably recycled to the water/steam generator unit (16) via line (21) to be reused for the azeotropic distillation. This prevents the accumulation of an unproductive water phase in the reaction zone. The organic phase from (17) is returned to the reaction zone via line (23).

The water used for the azeotropic distillation in unit (13) may be liquid water or steam and is supplied by unit (16) as discussed above. Preferably most of the water is recycled from the liquid-liquid separators (17) and (18). Depending on the water solubility in the crude aldehyde stream, the amount of water purge taken from unit (16) as line (22), and/or water lost in vents, a water makeup to unit (16) may be needed. The water should be de-aerated (oxygen free) and demineralized, distilled, or from a reverse osmosis or similar pre-treatment system and halide-free. Additives may be used such as buffers to maintain suitable pH values (preferably between 4.5 and 9 and most preferably between 6.0 and 7.5.). The buffers suitable for this application are preferably not salts to avoid fouling of the azeotropic vaporizer thus preference is given to the water soluble weakly basic amine buffers used in U.S. Pat. No. 10,131,608. The buffer can advantageously be premixed and added to the water (e.g., in unit (16)) prior to being introduced to the azeotropic vaporizer or as a separate stream.

The amount of optional weakly basic amine to be added can be calculated based on the pH drop observed between the feed of water to the azeotropic vaporizer (13) (i.e., in stream (14)) compared to the pH of the water leaving the L-L separator (17) either in stream (21) or (22). A pH drop greater than 0.5 units indicates that significant acid is being removed thus the feed should be increased. A drop of less than 0.1 units suggests that the feed can be reduced to save the cost of the amine additive.

In a preferred embodiment, a water purge (22) may also be used to remove water soluble impurities from the system such as acids, halides (from raw material feeds), and the like. In a preferred embodiment, a weakly basic amine is present in the water from the water reservoir (16) thus the purge will remove acidic impurities neutralized by the amine additive. The amount of flow in stream (22) is generally low and can be used to moderate the pH drop observed between streams (14) and (21). Due to environmental considerations, the flow is preferably sufficient to keep the pH drop described above below 0.5 pH units without excessive amounts of amine additive being used.

When the process of this invention is conducted as described hereinabove, then a catalyst recycle stream is obtained with a controlled quantity, preferably, a reduced quantity of heavies, as compared against a baseline process (comparative process) identical to the process of this invention with the exception that no water is added to the azeotropic vaporizer. For the baseline process refer to FIG. 1, which illustrates crude product feed to the vaporizer, an overhead gas stream from the vaporizer to the condenser, and gas overhead output from the condenser, without the use of added water to generate an azeotrope in the vaporizer and with recycle of any portion thereof back to the vaporizer. In the baseline process, especially when the temperature of the vaporizer must be lowered to accommodate the lower stability of the transition metal-organophosphorous ligand complex catalyst and free organophosphorous ligand, the heavies do not leave the vaporizer in sufficient quantity, and as such a larger quantity of heavies by-products is detrimentally recycled to the hydroformylation step. The process of this invention removes more heavies by-products in the azeotropic vaporizer overhead gas stream, allowing for beneficial operation of the vaporization process at a lower temperature for catalyst longevity and use of a conventional water-cooling condenser without a costly coolant and refrigeration apparatus. The present invention also allows operating the vaporization process at lower temperature which may slow the formation of heavies without a reduction in the aldehyde vaporization rate due to operating at a lower temperature or requiring a lower system pressure.

The use of the optional weakly amine additive in the water added to the azeotropic distillation process mitigates the issue of any acid-catalyzed phosphite degradation and acid-catalyzed heavies formation during the vaporization process. By neutralizing and removing these acids, these side reactions should be reduced.

In the case where triarylphosphines are used, the lower temperatures in the azeotropic vaporizer may reduce the amount of triarylphosphine volatilized out of the reaction fluid. Triarylphosphines such as triphenylphosphine have a low but not negligible vapor pressure thus may be volatilized during vaporization of higher molecular weight aldehydes (see WO2022180394). The triarylphosphines do not appear to form azeotropes thus the present invention can reduce the vaporization temperature needed to remove aldehyde heavies while also reducing the loss of triarylphosphine ligand. The triarylphosphine ligand can have detrimental effects on downstream liquid-phase hydrogenation systems thus reduced volatilization may give downstream benefits.

Objects and advantages of this invention are further illustrated by the following examples, which also further clarify the invention. The particular materials and amounts thereof, as well as other conditions and details, recited in these examples should not be used to limit this invention. Rather they are illustrative of the whole invention. Other embodiments of the invention will be readily apparent to those skilled in the art from a consideration of this specification or practice of the invention as disclosed herein. Examples of the invention are numbered; while comparative samples, which are not examples of the invention, are designated alphabetically.

### Example 1 for C9 Aldehyde Mixture

The azeotrope experiments were performed in batch mode with both the organic phase and the aqueous phase mixed and heated simultaneously. For the experiment, 150 g of the organic phase and 50 g of distilled water were placed in the reboiler. The organic phase consisted of the rhodium-ligand catalyst, C8 olefins, C9 aldehydes, C9 aldehyde dimers, trimers and heavier by-products (all combined termed as heavies) and trace quantity of C4-C7 olefins and alkanes. The ligand used in this experiment was tris-(2,4-ditertbutylphenyl)phosphite at approximately 0.25 wt %. The flask was set on a heating mantle and the pressure of the experiment was set at 1 atm absolute. The reboiler is connected to a spinning band column and the overhead of the spinning band column is connected to a condenser where the condenser cooling media is circulated at 5 °C. Stirring is started on the organic and aqueous phase mixture as the reboiler is heated to 100 °C-110 °C, which is slightly higher than the azeotropic temperature of the aldehyde-water system measured at around 99 °C. The overhead condensate was collected as the liquid mixture in reboiler is being heated and contained both organic and aqueous phase which were separated using a separatory funnel, after which the phases were weighed and analyzed for understanding the composition of the condensate.

### Composition Data for the Organic Phase:

Feed

| Component | wt % |
|---|---|
| Lights (C4-C7 hydrocarbons) | 1.29 |
| C8 olefin | 12.07 |
| C9 hydrocarbon | 0.61 |
| C9 aldehyde | 67.64 |
| Heavies | 18.46 |

Organic Condensate

| Component | wt % |
|---|---|
| Lights (C4-C7 hydrocarbons) | 4.39 |
| C8 olefin | 46.98 |
| C9 hydrocarbon | 1.98 |
| C9 aldehyde | 45.96 |
| Heavies | 0.69 |

### Condensate Weight Data:

| | Aqueous layer (g) | Organic layer (g) |
|---|---|---|
| Condensate | 36.4 | 36.8 |

The heavies vaporized/aldehyde vaporized is 0.69/45.96 or about 0.015 g/g in the azeotropic case which is higher than the heavies production rate of ~0.001-0.005 g/g thus the removal of heavies is higher than the make-rate.

### Example 2 for C9 Aldehyde Mixture

This experiment was conducted in a similar manner to example 1, where 100 g of the organic phase and 100 g of the aqueous phase were added to the reboiler. The pressure was set at 0.53 atm absolute and the reboiler was heated to 82 °C-88 °C to test the azeotrope formation at reduced pressure.

### Composition Data for the Organic Phase:

Feed

| Component | wt % |
|---|---|
| Lights (C4-C7 hydrocarbons) | 0.93 |
| C8 olefin | 6.9 |
| C9 hydrocarbon | 0.6 |
| C9 aldehyde | 68.34 |
| Heavies | 23.29 |

Organic Condensate

| Component | wt % |
|---|---|
| Lights (C4-C7 hydrocarbons) | 1.43 |
| C8 olefin | 16.74 |
| C9 hydrocarbon | 1.28 |
| C9 aldehyde | 79.17 |
| Heavies | 1.38 |

### Condensate Weight Data:

| | Aqueous layer (g) | Organic layer (g) |
|---|---|---|
| Condensate | 61.8 | 34.8 |

The heavies vaporized/aldehyde vaporized is 1.38/79.17 or about 0.023 g/g in the azeotropic case which is higher than Example 1 and the heavies production rate of ~0.001-0.005 g/g thus the removal of heavies is higher than the make-rate.

### Example 3 for C9 Aldehyde Mixture with Higher Amount of Heavies

This experiment was conducted in a similar manner to example 1, where 100 g of the organic phase and 300 g of the aqueous phase were added to the reboiler. The organic phase in this case contains more heavies and represents a typical stream from a primary separation unit operation after separating the aldehyde product. The pressure was set at 1 atm absolute and the reboiler was heated to 100 °C-110 °C, which is slightly higher than the azeotropic temperature of the aldehyde-water system which should be at 99 °C.

### Composition Data for the Organic Phase:

Feed

| Component | wt % |
|---|---|
| Lights (C4-C7 hydrocarbons) | 0.53 |
| C8 olefin | 13.7 |
| C9 hydrocarbon | 0.468 |
| C9 aldehyde | 29.04 |
| Heavies | 56.32 |

Organic Condensate

| Component | wt % |
|---|---|
| Lights (C4-C7 hydrocarbons) | 1.65 |
| C8 olefin | 49.59 |
| C9 hydrocarbon | 1.40 |
| C9 aldehyde | 42.25 |
| Heavies | 5.16 |

### Condensate Weight Data:

| | Aqueous layer (g) | Organic layer (g) |
|---|---|---|
| Condensate | 45.4 | 19.9 |

The heavies vaporized/aldehyde vaporized is 5.16/42.25 or about 0.12 g/g in the azeotropic case which is higher than Examples 1 and 2 and the heavies production rate of ~0.001-0.005 g/g thus the removal of heavies is higher than the make-rate. As the heavies content builds in the hydroformylation fluid, the azcotropic vaporizer is able to remove more heavies under the same conditions due to the higher incoming heavies concentration (i.e., the heavies azeotrope is enhanced). C9 aldehyde is still the main product from the azeotropic vaporizer so such a configuration can also increase the F/T ratio in the system.

Following table gives an insight into the amount of water needed for vaporizing aldehydes/all heavies from the three examples above—

| | Water | Aldehyde | |
|---|---|---|---|
| | (g) | (g) | Water/ aldehyde |
| Example | 36.40 | 16.91 | 2.15 |
| 1 | | | |
| Example | 61.80 | 27.55 | 2.24 |
| 2 | | | |
| Example | 45.4 | 8.41 | 5.4 |
| 3 | | | |

| | Water | Heavies | |
|---|---|---|---|
| | (g) | (g) | Water/ Heavies |
| Example | 36.40 | 0.26 | 142.68 |
| 1 | | | |
| Example | 61.80 | 0.48 | 128.28 |
| 2 | | | |
| Example | 45.4 | 1.03 | 44.19 |
| 3 | | | |

The ratio calculated in the table above between water and the organic phase (aldehyde or heavies) refers to the amount of water evaporated per unit mass of the organic evaporated. For the aldehyde case, the water/aldehyde ratio stays in the range of 2.15-2.24 given the similar starting aldehyde feed composition as seen from examples 1 and 2. For example 3, the water/aldehyde ratio is 5.4 as more water is needed when the aldehyde concentration is lower as compared to example 1 and 2. This ratio would change with parameters such as temperature, pressure, varying carbon length, feed composition, branching in aldehyde to name a few.

For the heavies case, the water/heavies ratio changes significantly between the example 1, 2 and 3. Between example 1 and 2 which have similar feed composition, lower operating pressure in example 2 favors lower water to heavies ratio of 128.28 as compared to 142.68 in example 1. The water to heavies ratio is reduced further to 44.19 when the heavies composition is increased as shown in example 3 when compared to examples 1 and 2. This reduces the amount of water needed in the overall process and makes the azeotropic vaporizer well suited to accept concentrated heavies streams from a preliminary separation unit operation. This also highlights the fact that similar to the aldehyde case, the higher heavies concentration reduces the amount of water needed for the azeotrope and vice versa. Also similar to the aldehyde case, the water/heavies ratio depends on temperature, pressure, varying carbon length, feed composition, branching in aldehyde to name a few.

Example 3 also shows that the present invention is well suited for highly concentrated catalyst streams such as generated from conventional purge streams such that the purged stream is treated with an intensive distillation process prior to being sent off to precious metal recovery. The present invention will facilitate the recovery of residual aldehyde product and may facilitate the operation of the intensive distillation (e.g., wiped film evaporator) by reducing viscosity and providing an internal stripping gas. In this scheme, referring to FIG. 3, the purge stream is stream (25b) and there may not be a recycle line from the azeotropic vaporizer back to the reactor.

In a continuous azeotropic vaporizer the amount of water needed to achieve a particular F/T and aldehyde production rate would be tuned according to the water/aldehyde ratio mentioned above for the particular system. This ratio will be the minimum amount of water needed for the azeotropic vaporizer and any excess amount of water should remain in the tails stream. Based on the amount of water, the amount of heavies vaporized can be calculated from the water/heavies ratio. If the heavies rate of removal from azeotropic vaporizer is higher than the heavies production rate, the heavies concentration in the reactors will decrease. This decrease would reduce the heavies concentration coming into the azeotropic vaporizer, reducing the heavies removal rate. This process would happen till the heavies removal rate and the production rate are equal.

In summary, it is seen that the process of the invention provides for greater heavies removal in the vaporizer overhead gas stream, with controlled quantity of heavies in the liquid recycle stream to the hydroformylation step, at a lower temperature of operation of the vaporizer for greater catalyst stability.

1. A process of controlling heavies in a catalyst recycle stream, the process comprising:
(a) removing a crude product stream from a hydroformylation reaction zone comprising one or more aldehyde products, one or more heavies by-products, a transition metal-organophosphorous ligand complex catalyst, one or more unconverted reactants, and one or more inert lights;
(b) providing a water stream;
(c) combining the water stream from step (b) with the stream from step (a) into an azeotropic vaporizer;
(d) removing from the vaporizer an overhead gas stream comprising one or more aldehyde products, one or more unconverted reactants, one or more inert lights, a portion of the water, and a portion of the heavies by-products, and feeding said overhead gas stream into a condenser;
(e) removing from the condenser an overhead gas stream comprising one or more unconverted reactants, a portion of the added water, and one or more inert lights;
(f) recovering a liquid stream from the condenser comprising the aldehyde product, heavies by-products, and water;
(g) separating the organic components from the water from step (f) in a liquid-liquid separation zone to recover the crude aldehyde product as the top layer and an aqueous phase as the bottom layer; and
(h) removing as a tails stream from the vaporizer, a liquid recycle catalyst stream comprising the transition metal-organophosphorous ligand complex catalyst, the balance of the aldehyde product and the balance of the heavies by-products to be sent to the reaction zone wherein at least 25% of the aldehyde product recovered in step (d) is removed as a water azeotrope.

2. The process of claim 1 where the crude product stream from step (a) is passed to a preliminary product/catalyst separation process prior to step (c).

3. The process of claim 2 wherein the preliminary product/catalyst separation process involves a vaporization process wherein the top stream comprises a first crude aldehyde product stream and the bottom stream comprises a transition metal-organophosphorous ligand complex catalyst and heavies, and wherein at least a portion of the bottom stream is then fed to step (c).

4. The process of claim 2 wherein the preliminary product/catalyst separation process involves a membrane separation process resulting in a retentate portion and a permeate portion, wherein the retenate comprises a transition metal-organophosphorous ligand complex catalyst, some aldehyde product and heavies stream which can be recycled to the reaction zone, and the permeate comprises aldehyde product, at least some heavies, unconverted reactants, inert lights and some catalyst components at least a portion of which is then fed to step (c).

5. The processes of claim 1 wherein at least a portion of the bottom water phase from step (g) is recycled to step (b).

6. The processes of claim 1 wherein the tails stream from step (h) is cooled and sent to a liquid-liquid separation zone to recover the catalyst-containing organic phase as the top layer which can be recycled to the reaction zone, and an aqueous phase as the bottom layer.

7. The process of claim 6 wherein at least a portion of the bottom water phase is recycled to step (b).

8. The process of claim 3 wherein the majority of the total aldehyde product is separated in the preliminary product/catalyst separation zone.

9. The process of claim 1 wherein at least 90% of the product recovered in step (d) was removed as a water azeotrope.

10. The process of claim 1 wherein the amount of product removed as the azeotrope is controlled by adjusting the amount of water added in step (c)

11. The process of claim 1 wherein a buffer is added to the water feed of step (b).

12. The process of claim 11 wherein the buffer is a weakly basic amine.

13. The process of claim 1 wherein the water is added as steam.

14. The process of claim 1 wherein the water is added as liquid water.

15. The process of claim 1 wherein the azeotropic vaporizer is a stripping gas vaporizer.

16. The process of claim 1 wherein the wherein the transition metal-organophosphorous ligand complex catalyst comprises one or more of the following organophosphites:
a) Methyl [3,3′-di-t-butyl-5,5′-dimethoxy-1,1′biphenyl-2,2′-diyl]phosphite represented by Formula W: 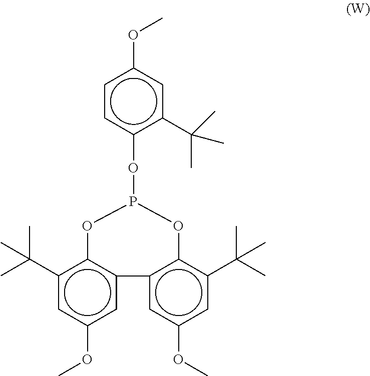
and (b)tris-(2,4-di-tert-butylphenyl)phosphite represented by Formula X: 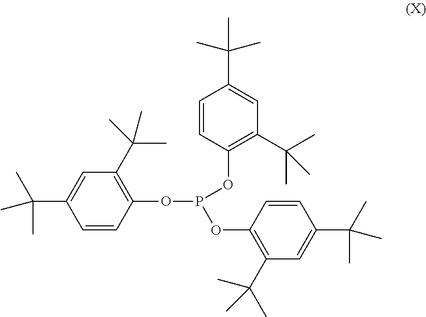 [SMILES: CC(C)(C)c1ccc(OP(Oc2ccc(C(C)(C)C)cc2C(C)(C)C)Oc2ccc(C(C)(C)C)cc2C(C)(C)C)c(C(C)(C)C)c1]
